(19) United States
(12) Patent Application Publication
(10) Pub. No.: US 2011/0257190 A1
(43) Pub. Date: Oct. 20, 2011

(54) CRYSTALLINE FORMS

(75) Inventors: Kenneth J. BORDEAU, Kintnersville, PA (US); Beverly C. LANGEVIN, II, Stewartsville, NJ (US); George Everett LEE, Somerville, NJ (US); Patricia LOGUE, Medford, NJ (US); Elizabeth SECORD, Langhorne, PA (US); Daniel SHERER, Berkley Heights, NJ (US)

(73) Assignee: SANOFI, Paris (FR)

(21) Appl. No.: 13/151,853

(22) Filed: Jun. 2, 2011

Related U.S. Application Data
(63) Continuation of application No. PCT/US2009/066489, filed on Dec. 3, 2009.
(60) Provisional application No. 61/119,866, filed on Dec. 4, 2008.

(51) Int. Cl. A61K 31/4985; A61K 31/519; A61P 9/00; A61P 29/00; C07D 487/02; A61P 35/00

(52) U.S. Cl. 514/249; 544/350

(57) ABSTRACT

The disclosure relates to crystalline forms of 2-[4-(7-ethyl-5H-pyrrolo[2,3-b]pyrazin-6-yl)-phenyl]-propan-2-ol, characterized by physico-chemical data described herein.

This application is a continuation of International Application No. PCT/US2009/066489, filed Dec. 3, 2009, which claims the benefit of priority of U.S. Provisional Application No. 61/119,866, filed Dec. 4, 2008, both of which are incorporated herein by reference.

### FIELD OF THE INVENTION

This invention is directed to novel forms of substituted azaindoles, their preparation, pharmaceutical compositions containing these compounds, and their pharmaceutical use in the treatment of disease states capable of being modulated by the inhibition of the protein kinases.

### BACKGROUND OF THE INVENTION

Compound I:

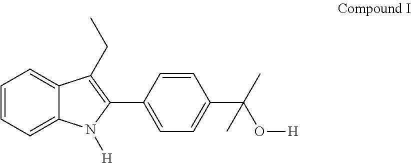

and the synthesis of this compound have been disclosed in the international patent application WO2008/033798. This compound is also known by the name: 2-[4-(7-ethyl-5H-pyrrolo[2,3-b]pyrazin-6-yl)-phenyl]-propan-2-ol. This compound is useful for inhibiting protein kinases, and in particular the Syk or spleen tyrosine kinase. The compound is also useful as a pharmaceutical for treating diseases such as arthritis and macular degeneration.

It has now been discovered that Compound I exists in novel crystalline forms which are particularly suitable for use in making pharmaceutical compositions. These forms can be prepared by efficient, economical and reproducible processes that are particularly suited to large-scale preparation.

### SUMMARY OF THE INVENTION

The present invention relates to particular forms of Compound I:

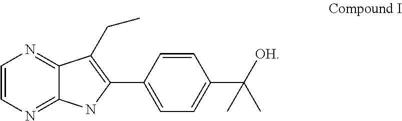

These forms can be obtained from solvents including but not limited to ethyl acetate, n-propanol, methyl tetrahydrofuran, TBME, methanol, n-butanol, isobutanol, toluene, amyl alcohol, t-amyl alcohol and ethyl formate. These crystalline forms are also obtained by sublimation. These crystalline forms are characterized by specific properties such as an ROD (x-ray powder diffraction) pattern, DSC (differential scanning calorimetry) behavior, a melting point, and Raman spectroscopy. These properties are shown below.

The invention is also directed to a pharmaceutical composition comprising Form A, Form B or Form C of 2-[4-(7-ethyl-5H-pyrrolo[2,3-b]pyrazin-6-yl)-phenyl]-propan-2-ol, and a method for using these crystalline forms of compound I for or treating or preventing a physiological condition related to Syk in a patient.

The invention is also directed to crystalline forms A, B, and C of 2-[4-(7-ethyl-5H-pyrrolo[2,3-b]pyrazin-6-yl)-phenyl]-propan-2-ol and processes for preparing Form A, B, and C.

### BRIEF DESCRIPTION OF THE DRAWINGS

FIG. 1 is an X-ray powder diffractogram of crystalline 2-[4-(7-ethyl-5H-pyrrolo[2,3-b]pyrazin-6-yl)-phenyl]-propan-2-ol Form A of the present invention.

FIG. 2 is a Raman spectrum of 2-[4-(7-ethyl-5H-pyrrolo[2,3-b]pyrazin-6-yl)-phenyl]-propan-2-ol Form A of the present invention.

FIG. 3 is a Differential Scanning calorimetry thermogram of 2-[4-(7-ethyl-5H-pyrrolo[2,3-b]pyrazin-6-yl)-phenyl]-propan-2-ol Form A of the present invention.

FIG. 4 is an X-ray powder diffractogram of crystalline 2-[4-(7-ethyl-5H-pyrrolo[2,3-b]pyrazin-6-yl)-phenyl]-propan-2-ol Form B of the present invention.

FIG. 5 is a Raman spectrum of 2-[4-(7-ethyl-5H-pyrrolo[2,3-b]pyrazin-6-yl)-phenyl]-propan-2-ol Form B of the present invention.

FIG. 6 is a Differential Scanning calorimetry—Thermal Gravimetric Analysis thermogram of 2-[4-(7-ethyl-5H-pyrrolo[2,3-b]pyrazin-6-yl)-phenyl]-propan-2-ol Form B of the present invention.

FIG. 7 is an X-ray powder diffractogram of crystalline 2-[4-(7-ethyl-5H-pyrrolo[2,3-b]pyrazin-6-yl)-phenyl]-propan-2-ol Form C of the present invention.

FIG. 8 is a Raman spectrum of 2-[4-(7-ethyl-5H-pyrrolo[2,3-b]pyrazin-6-yl)-phenyl]-propan-2-ol Form C of the present invention.

FIG. 9 is a Differential Scanning calorimetry—Thermal Gravimetric Analysis thermogram of 2-[4-(7-ethyl-5H-pyrrolo[2,3-b]pyrazin-6-yl)-phenyl]-propan-2-ol Form C of the present invention.

FIG. 10 is an overlay of X-ray powder diffractograms of crystalline 2-[4-(7-ethyl-5H-pyrrolo[2,3-b]pyrazin-6-yl)-phenyl]-propan-2-ol Form A (bottom), 2-[4-(7-ethyl-5H-pyrrolo[2,3-b]pyrazin-6-yl)-phenyl]-propan-2-ol Form B (middle), and 2-[4-(7-ethyl-5H-pyrrolo[2,3-b]pyrazin-6-yl)-phenyl]-propan-2-ol Form C (top) of the present invention.

FIG. 11 is an overlay of DSC-TGA thermograms of 2-[4-(7-ethyl-5H-pyrrolo[2,3-b]pyrazin-6-yl)-phenyl]-propan-2-ol Form A (top), 2-[4-(7-ethyl-5H-pyrrolo[2,3-b]pyrazin-6-yl)-phenyl]-propan-2-ol Form B (middle), and 2-[4-(7-ethyl-5H-pyrrolo[2,3-b]pyrazin-6-yl)-phenyl]-propan-2-ol Form C (bottom) of the present invention.

### ABBREVIATIONS

As used above, and throughout the description of the invention, the following abbreviations, unless otherwise indicated, shall be understood to have the following meanings

EtOAc ethyl acetate
n-PrOH 1-propanol
TBME tert-Butyl methyl ether
THF tetrahydrofuran
2-methylTHF 2-methyl-tetrahydrofuan

### DETAILED DESCRIPTION OF THE INVENTION

The synthesis of Compound I has been disclosed in the international patent application WO2008/033798. The present invention provides a process for the manufacture of the 2-[4-(7-ethyl-5H-pyrrolo[2,3-b]pyrazin-6-yl)-phenyl]-propan-2-ol of formula (I) in crystalline form, said process comprising the steps of dissolving 2-[4-(7-ethyl-5H-pyrrolo[2,3-b]pyrazin-6-yl)-phenyl]-propan-2-ol, under elevated temperature or at ambient temperature, in a suitable solvent or in a mixture of solvents and isolating the precipitated solid, for example by filtration or removal of the solvent.

In one aspect of the invention, the crystalline form of 2-[4-(7-ethyl-5H-pyrrolo[2,3-b]pyrazin-6-yl)-phenyl]-propan-2-ol described herein as 2-[4-(7-ethyl-5H-pyrrolo[2,3-b]pyrazin-6-yl)-phenyl]-propan-2-ol crystalline Form A exhibits an X-ray diffraction pattern comprising peaks at about 7.30, 9.09, 11.08, 11.47, 12.34, and 14.67 degrees 2-theta.

Another particular aspect of the invention is a process for preparing 2-[4-(7-ethyl-5H-pyrrolo[2,3-b]pyrazin-6-yl)-phenyl]-propan-2-ol crystalline Form A. To obtain 2-[4-(7-ethyl-5H-pyrrolo[2,3-b]pyrazin-6-yl)-phenyl]-propan-2-ol in crystalline Form A, 2-[4-(7-ethyl-5H-pyrrolo[2,3-b]pyrazin-6-yl)-phenyl]-propan-2-ol is dissolved in a suitable solvent or in a mixture of solvents, including but not limited to methanol, n-propanol, n-butanol, isobutanol, toluene, amyl alcohol, t-amyl alcohol, 2-methyl tetrahydrofuran, methyl ethyl ketone, isopropyl acetate, and water, and isolating the precipitated solid, for example by filtration or removal of the solvent by vacuum drying.

In another aspect of the invention the crystalline form of 2-[4-(7-ethyl-5H-pyrrolo[2,3-b]pyrazin-6-yl)-phenyl]-propan-2-ol described herein as 2-[4-(7-ethyl-5H-pyrrolo[2,3-b]pyrazin-6-yl)-phenyl]-propan-2-ol crystalline Form B exhibits an X-ray diffraction pattern comprising peaks at about 7.50, 8.45, 12.46, 13.11, 15.03, 16.90, and 17.78 degrees 2-theta.

Another particular aspect of the invention is a process for preparing 2-[4-(7-ethyl-5H-pyrrolo[2,3-b]pyrazin-6-yl)-phenyl]-propan-2-ol crystalline Form B. To obtain 2-[4-(7-ethyl-5H-pyrrolo[2,3-b]pyrazin-6-yl)-phenyl]-propan-2-ol in crystalline Form B, 2-[4-(7-ethyl-5H-pyrrolo[2,3-b]pyrazin-6-yl)-phenyl]-propan-2-ol is dissolved in a suitable solvent or in a mixture of solvents, including but not limited to methanol, n-propanol, n-butanol, isobutanol, toluene, amyl alcohol, t-amyl alcohol, 2-methyl tetrahydrofuran, methyl ethyl ketone, isopropyl acetate, and water, and isolating the precipitated solid, for example by filtration or removal of the solvent by vacuum drying.

Another particular aspect of the invention, a process for preparing 2-[4-(7-ethyl-5H-pyrrolo[2,3-b]pyrazin-6-yl)-phenyl]-propan-2-ol Form B comprises subliming 2-[4-(7-ethyl-5H-pyrrolo[2,3-b]pyrazin-6-yl)-phenyl]-propan-2-ol crystalline Form A by heating above about 180° C.

Another particular aspect of the invention is a process for preparing 2-[4-(7-ethyl-5H-pyrrolo[2,3-b]pyrazin-6-yl)-phenyl]-propan-2-ol Form B comprising subliming 2-[4-(7-ethyl-5H-pyrrolo[2,3-b]pyrazin-6-yl)-phenyl]-propan-2-ol crystalline Form C by heating above about 180° C.

In another aspect of the invention, the crystalline form of 2-[4-(7-ethyl-5H-pyrrolo[2,3-b]pyrazin-6-yl)-phenyl]-propan-2-ol described herein as 2-[4-(7-ethyl-5H-pyrrolo[2,3-b]pyrazin-6-yl)-phenyl]-propan-2-ol crystalline form C exhibits an X-ray diffraction pattern comprising peaks at about 6.58, 7.35, 8.23, 9.01, 11.87, and 13.12 degrees 2-theta.

Another particular aspect of the invention is a process for preparing 2-[4-(7-ethyl-5H-pyrrolo[2,3-b]pyrazin-6-yl)-phenyl]-propan-2-ol crystalline Form C. To obtain 2-[4-(7-ethyl-5H-pyrrolo[2,3-b]pyrazin-6-yl)-phenyl]-propan-2-ol in crystalline Form C, 2-[4-(7-ethyl-5H-pyrrolo[2,3-b]pyrazin-6-yl)-phenyl]-propan-2-ol is dissolved in a suitable solvent or in a mixture of solvents, including but not limited to 2-methyl THF, TBME, and ethyl formate, and isolating the precipitated solid, for example by filtration or removal of the solvent by vacuum drying.

2-[4-(7-Ethyl-5H-pyrrolo[2,3-b]pyrazin-6-yl)-phenyl]-propan-2-ol exhibits useful pharmacological activity and accordingly can be incorporated into pharmaceutical compositions and used in the treatment of patients suffering from certain medical disorders.

2-[4-(7-Ethyl-5H-pyrrolo[2,3-b]pyrazin-6-yl)-phenyl]-propan-2-ol inhibits or blocks kinase catalytic activity according to tests described in the literature and described in international patent application WO2008/033798, which tests' results are believed to correlate to pharmacological activity in humans and other mammals. Thus, in a further embodiment, the present invention provides 2-[4-(7-ethyl-5H-pyrrolo[2,3-b]pyrazin-6-yl)-phenyl]-propan-2-ol of the invention and compositions containing 2-[4-(7-ethyl-5H-pyrrolo[2,3-b]pyrazin-6-yl)-phenyl]-propan-2-ol of the invention for use in the treatment of a patient suffering from, or subject to, conditions which can be ameliorated by the administration of protein kinase inhibitors (e.g. Syk, FAK, KDR or Aurora2). For example, 2-[4-(7-ethyl-5H-pyrrolo[2,3-b]pyrazin-6-yl)-phenyl]-propan-2-ol is useful in the treatment of inflammatory diseases, for example asthma: inflammatory dermatoses (e.g. psoriasis, dermatitis herpetiformis, eczema, necrotizing and cutaneous vasculitis, bullous disease); allergic rhinitis and allergic conjunctivitis; joint inflammation, including arthritis, rheumatoid arthritis and other arthritic conditions such as rheumatoid spondylitis, gouty arthritis, traumatic arthritis, rubella arthritis, psoriatic arthritis and osteoarthritis. 2-[4-(7-ethyl-5H-pyrrolo[2,3-b]pyrazin-6-yl)-phenyl]-propan-2-ol is also useful in the treatment of Chronic Obstructive Pulmonary Disease (COPD), acute synovitis, autoimmune diabetes, autoimmune encephalomyelitis, colitis, atherosclerosis, peripheral vascular disease, cardiovascular disease, multiple sclerosis, restenosis, myocarditis, B cell lymphomas, systemic lupus erythematosus, graft versus host disease and other transplant associated rejection events, cancers and tumors (such as colorectal, prostate, breast, thyroid, colon and lung cancers), acute macular degeneration, and inflammatory bowel disease. Additionally, 2-[4-(7-ethyl-5H-pyrrolo[2,3-b]pyrazin-6-yl)-phenyl]-propan-2-ol is useful as a tumor anti-angiogenic agent. Furthermore, 2-[4-(7-ethyl-5H-pyrrolo[2,3-b]pyrazin-6-yl)-phenyl]-propan-2-ol is useful as an agent to control tumor cells.

A special embodiment of the therapeutic methods of the present invention is the treating of joint inflammation.

Another special embodiment of the therapeutic methods of the present invention is the treating of rheumatoid arthritis.

A special embodiment of the therapeutic methods of the present invention is the treating of cancers, tumors and other proliferative disorders.

Another special embodiment of the therapeutic methods of the present invention is the treating of cancers involving liquid tumors.

Another special embodiment of the therapeutic methods of the present invention is the treating of mantle cell lymphoma.

Another special embodiment of the therapeutic methods of the present invention is the treating of AMD, retinal edema, diabetic retinopathy, etc.

Yet another special embodiment of the therapeutic methods of the present invention is the treating of disorders by inhibition of angiogenesis.

According to a further feature of the invention there is provided a method for the treatment of a human or animal patient suffering from, or subject to, conditions which can be ameliorated by the administration of a protein kinase inhibitor (e.g. Syk, FAK, KDR or Aurora2) for example conditions as hereinbefore described, which comprises the administration to the patient of an effective amount of Form A of the invention or a composition containing Form A of the invention. “Effective amount” is meant to describe an amount of compound of the present invention effective in inhibiting the catalytic activity a protein kinase, such as Syk, FAK, KDR or Aurora2, and thus producing the desired therapeutic effect.

References herein to treatment should be understood to include prophylactic therapy as well as treatment of established conditions.

The present invention also includes within its scope pharmaceutical compositions comprising 2-[4-(7-ethyl-5H-pyrrolo[2,3-b]pyrazin-6-yl)-phenyl]-propan-2-ol in association with a pharmaceutically acceptable carrier or excipient.

2-[4-(7-Ethyl-5H-pyrrolo[2,3-b]pyrazin-6-yl)-phenyl]-propan-2-ol of the invention may be administered by any suitable means. In practice a compound of the present invention may generally be administered parenterally, topically, rectally, orally or by inhalation; especially by the oral route or by inhalation.

Compositions according to the invention may be prepared according to the customary methods, using one or more pharmaceutically acceptable adjuvants or excipients. The adjuvants comprise, inter alia, diluents, sterile aqueous media and the various non-toxic organic solvents. The compositions may be presented in the form of tablets, pills, granules, powders, aqueous solutions or suspensions, injectable solutions, elixirs or syrups, and can contain one or more agents chosen from the group comprising sweeteners, flavourings, colourings, or stabilizers in order to obtain pharmaceutically acceptable preparations. The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulfate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

For parenteral administration, emulsions, suspensions or solutions of the products according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are used. The solutions of the salts of the products according to the invention are especially useful for administration by intramuscular or subcutaneous injection. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation or microfiltration.

For topical administration, gels (water or alcohol based), creams or ointments containing compounds of the invention may be used. Compounds of the invention may also be incorporated in a gel or matrix base for application in a patch, which would allow a controlled release of compound through the transdermal barrier.

For administration by inhalation compounds of the invention may be dissolved or suspended in a suitable carrier for use in a nebulizer or a suspension or solution aerosol, or may be absorbed or adsorbed onto a suitable solid carrier for use in a dry powder inhaler.

Solid compositions for rectal administration include suppositories formulated in accordance with known methods and containing at least one compound of the invention.

### Topical Eye Delivery

Compositions suitable for topical administration are known to the art (see, for example, U.S. Patent Application 2005/0059639). In various embodiments, compositions of a compound having applications in a method of the present invention can comprise a liquid comprising a compound in solution, in suspension, or both. As used herein, liquid compositions include gels. Preferably the liquid composition is aqueous. Alternatively, the composition can take form of an ointment. In a preferred embodiment, the composition is an in situ gellable aqueous composition, more preferably an in situ gellable aqueous solution. Such a composition can comprise a gelling agent in a concentration effective to promote gelling upon contact with the eye or lacrimal fluid in the exterior of the eye. Aqueous compositions of the invention have ophthalmically compatible pH and osmolality.

Topical application (dropped into the eye) of a compound having applications in a method of the present invention may permit one to avoid most of the systemic side effects, but for effectiveness it is essential that the compound or compounds be actively transported across the cornea at sufficient levels to reach the proper receptor site.

Typically, administration of a compound having applications in a method of the present invention involves preparing an ointment (such as petrolatum) or gel with a conventional pharmaceutical carrier (such as carbopol) and topically administering the gel composition. The amount of the compound in the composition should be from about 0.25% by weight to about 5% by weight for an eye drop composition, preferably from about 0.5% by weight to about 2.0% by weight. The important point is not the dose amount, but simply that it be an amount that is effective in treating, preventing or ameliorating macular degeneration and yet not be so strong as to provide eye irritation or side effects. Generally, amounts within the ranges herein specified are satisfactory. Moreover, one of ordinary skill in the art can readily determine the appropriate ranges using routine laboratory techniques.

The carrier for the eye drop composition may be an isotonic eye treatment carrier buffered to a pH of from about 4 to about 8, and typically it will contain small amounts of conventional wetting agents and anti-bacterial agents. The preferred pH is within the range of from about 6.8 to about 7.8 and contains sufficient sodium chloride or equivalent to be isotonic. Anti-bacterial agents, when they are included, may be within the range of from about 0.004% (W/V) to about 0.02% (W/V) of the composition.

A compound having applications in a method of the present invention may be incorporated into various ophthalmic gel formulations for delivery to the eye. In order to form sterile ophthalmic ointment formulations, the compound may be combined with a preservative in an appropriate vehicle, such as mineral oil, liquid lanolin, or white petrolatum. Sterile ophthalmic gel formulations may be prepared by suspending a compound having applications in a method of the present invention in a hydrophilic base prepared from a combination of carbopol-940 (a carboxy vinyl polymer available from the B. F. Goodrich Company) according to published formulations for analogous ophthalmic preparations. Preservatives and tonicity agents can also be incorporated.

Subtenon delivery of a compound having applications in a method of the present invention also can be used to treat, prevent or ameliorate macular degeneration. A particular example of such delivery is set forth in U.S. Pat. No. 6,413,245, which discloses a method and apparatus for delivering a drug formulation to a human eye. The method includes the steps of inserting the apparatus below the Tenon's capsule and above the sclera at a point posterior to the limbus of the eye and injecting the drug formulation to form a drug depot on an outer surface of the sclera. The apparatus includes a cannula having a distal portion, a proximal portion, and a bend separating the distal portion and the proximal portion. The distal portion has a radius of curvature substantially equal to a radius of curvature of the globe of the eye. A tangent of the distal portion at the bend is disposed at an angle no more than about 56 degrees with respect to the proximal portion. Other subtenon delivery methods and apparati known to one of ordinary skill in this art readily applications herein.

### Intravitreal Delivery

The composition can comprise an ophthalmic depot formulation comprising a compound having applications in a method of the present invention for subconjunctival administration (different than intravitreal). The microparticles comprising the compound can be embedded in a biocompatible pharmaceutically acceptable polymer or a lipid encapsulating agent. The depot formulations may be adapted to release all or substantially all the compound over an extended period of time. The polymer or lipid matrix, if present, may be adapted to degrade sufficiently to be transported from the site of administration after release of all or substantially all the compound. The depot formulation can be a liquid formulation, comprising a pharmaceutical acceptable polymer and a dissolved or dispersed active agent. Upon injection, the polymer forms a depot at the injections site, e.g. by gelifying or precipitating. The composition can comprise a solid article that can be inserted in a suitable location in the eye, such as between the eye and eyelid or in the conjuctival sac, where the article releases the compound. Solid articles suitable for implantation in the eye in such fashion generally comprise polymers and can be bioerodible or non-bioerodible.

The percentage of active ingredient in the compositions of the invention may be varied, it being necessary that it should constitute a proportion such that a suitable dosage shall be obtained. Obviously, several unit dosage forms may be administered at about the same time. The dose employed will be determined by the physician, and depends upon the desired therapeutic effect, the route of administration and the duration of the treatment, and the condition of the patient. In the adult, the doses are generally from about 0.001 to about 50, preferably about 0.001 to about 5, mg/kg body weight per day by inhalation, from about 0.01 to about 100, preferably 0.1 to 70, more especially 0.5 to 10, mg/kg body weight per day by oral administration, and from about 0.001 to about 10, preferably 0.01 to 1, mg/kg body weight per day by intravenous administration. In each particular case, the doses will be determined in accordance with the factors distinctive to the subject to be treated, such as age, weight, general state of health and other characteristics which can influence the efficacy of the medicinal product.

The compounds according to the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active product may be administered orally 1 to 4 times per day. Of course, for some patients, it will be necessary to prescribe not more than one or two doses per day.

Synthesis of 2-[4-(7-ethyl-5H-pyrrolo[2,3-b]pyrazin-6-yl)-phenyl]-propan-2-ol has been disclosed in international patent application WO2008/033798.

### Example 1

### Preparation of crystalline 2-[4-(7-Ethyl-5H-pyrrolo[2,3-b]pyrazin-6-yl)-phenyl]-propan-2-ol Form A

Dissolve 2-[4-(7-Ethyl-5H-pyrrolo[2,3-b]pyrazin-6-yl)-phenyl]-propan-2-ol (1.002 kg) in n-propanol (16 L) by heating to 93° C.±3° C. A true solution in the vessel should be ensured with no precipitation on the vessel walls above the solution line. Maintaining a jacket temperature equal to or below the solution temperature minimizes precipitation on the vessel walls. Begin a cooling ramp based on the solution temperature at a rate of 30° C./h. A cooling rate between 12° C./h and 60° C./h is sufficient to produce Form A. The onset of precipitation should occur between 65° C. and 72° C. to obtain Form A (controlled by concentration and cooling rate). After precipitation has occurred, the cooling ramp is switched from batch temperature control to jacket temperature control using the same rate. Upon reaching 0° C., maintain the temperature for at least one hour before collecting the precipitate by filtration, wash with pre-cooled n-propanol (1 L), and dry at 70° C. under vacuum with nitrogen purge to yield 2-[4-(7-Ethyl-5H-pyrrolo[2,3-b]pyrazin-6-yl)-phenyl]-propan-2-ol Form A as a crystalline solid (yield 887.9 g, 88.6%).

### Example 2

### Preparation of crystalline 2-[4-(7-Ethyl-5H-pyrrolo[2,3-b]pyrazin-6-yl)-phenyl]-propan-2-ol Form B

Dissolve 2-[4-(7-Ethyl-5H-pyrrolo[2,3-b]pyrazin-6-yl)-phenyl]-propan-2-ol (200 g) in n-propanol (2.67 L) by heating to 85° C. at 60° C./h with further heating to 94° C. at 6° C./h. To minimize precipitation of solids above the solvent line keep the solution temperature close to the jacket temperature. Ensure a true solution in the vessel with no precipitation on the vessel walls above the solution line. Begin a three stage cooling ramp, from 94° C. to 68° C. at a rate of 1.63° C./h, from 68° C. to 50° C. at a rate of 12° C./h and from 50° C. to 0° C. at 25° C./h. The onset of precipitation should occur above 82° C. to obtain Form B (controlled by concentration and cooling rate). Upon reaching 0° C., maintain the temperature for at least one hour before collecting the precipitate by filtration, washing with pre-cooled n-propanol (200 mL), and drying at 70° C. under vacuum with nitrogen purge to yield 2-[4-(7-Ethyl-5H-pyrrolo[2,3-b]pyrazin-6-yl)-phenyl]-propan-2-ol Form B as a crystalline solid (yield 169.6 g, 84.8%).

### Example 5

### Preparation of crystalline 2-[4-(7-Ethyl-5H-pyrrolo[2,3-b]pyrazin-6-yl)-phenyl]-propan-2-ol Form C

Dissolve 2-[4-(7-Ethyl-5H-pyrrolo[2,3-b]pyrazin-6-yl)-phenyl]-propan-2-ol (52.5 mg) in methyltetrahydrofuran (1.3 mL) by heating to 80° C. Begin a cooling ramp from 80° C. to 20° C. at a rate of 12° C./h. The onset of precipitation should occur at 24° C. to obtain Form C. Upon reaching 20° C., maintain the temperature for at least one hour before collecting the precipitate by filtration and drying under vacuum to yield 2-[4-(7-Ethyl-5H-pyrrolo[2,3-b]pyrazin-6-yl)-phenyl]-propan-2-ol Form C as a crystalline solid.

The compounds of the invention are analyzed by the following analytical methods.

### X-Ray Powder Diffractometry

X-ray powder diffractometry is performed on a Siemens-Bruker D5000 diffractometer, using the parafocusing Bragg-Brentano (theta-two-theta)-type geometry. The compound of the invention, as a powder, is deposited on a single-crystal silicon wafer, cut according to the (510) crystallographic orientation. Copper K-alpha radiation (1.54056 angstroms), emitted from a copper anticathode tube (45 kV/40 mA) is used as the x-ray source, with Cu K-beta radiation filtered out using a reflected beam monochromator. A scintillation counter is used for detection. A divergence slit of 0.6 mm, an anti-scatter slit of 0.6 mm, a monochromator slit of 0.1 mm, and detector slit of 0.6 mm are used. The diffraction pattern is obtained using the following conditions: at least 2.0 to 30.0 degree scan in angle 2-theta, 1.0 second count time per step, 0.02 degree step size, under ambient conditions of pressure, temperature, and relative humidity except as noted. Above ambient temperatures were achieved by heating the sample at a linear rate of 0.03 to 0.06° C./second.

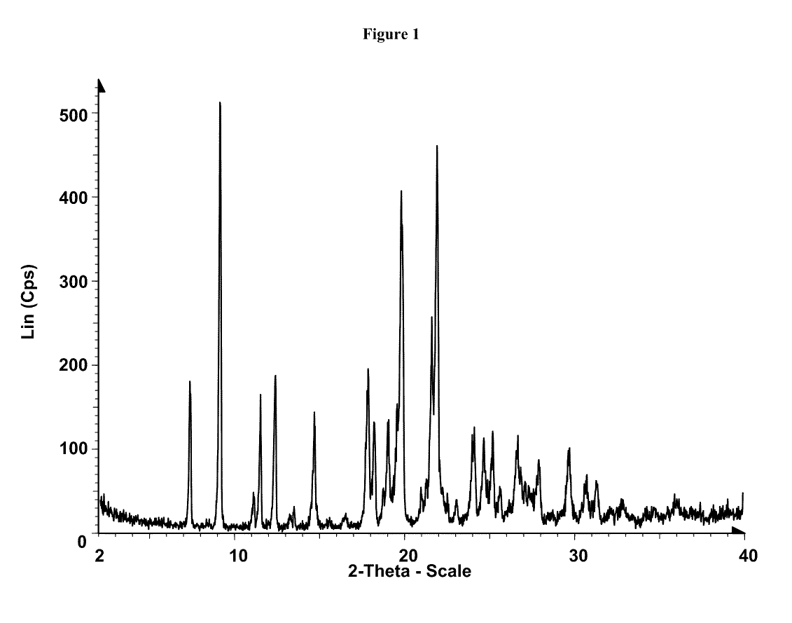

The XRPD spectra (FIG. 1) confirmed that the 2-[4-(7-ethyl-5H-pyrrolo[2,3-b]pyrazin-6-yl)-phenyl]-propan-2-ol Form A was crystalline.

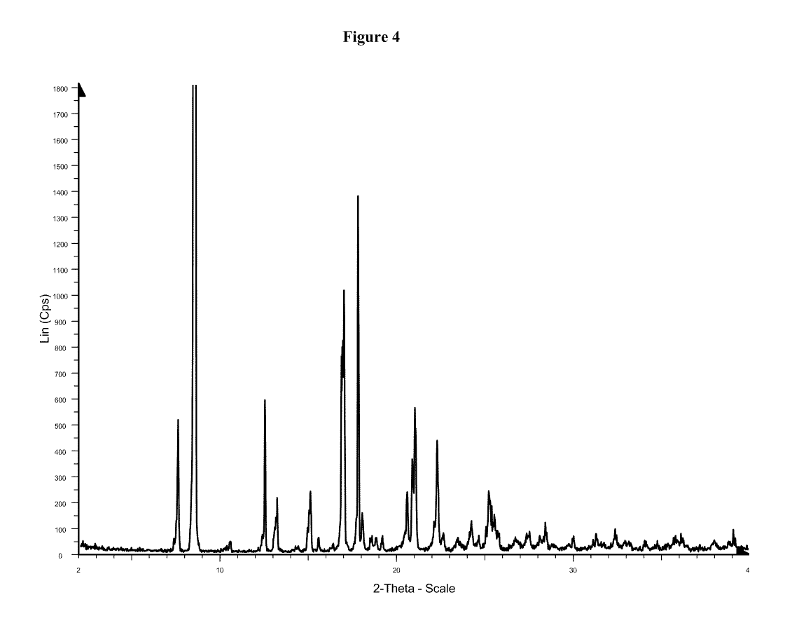

Based on the XRPD patterns, the 2-[4-(7-ethyl-5H-pyrrolo[2,3-b]pyrazin-6-yl)-phenyl]-propan-2-ol crystalline Form B (FIG. 4) has a unique XRPD pattern compared to Form A.

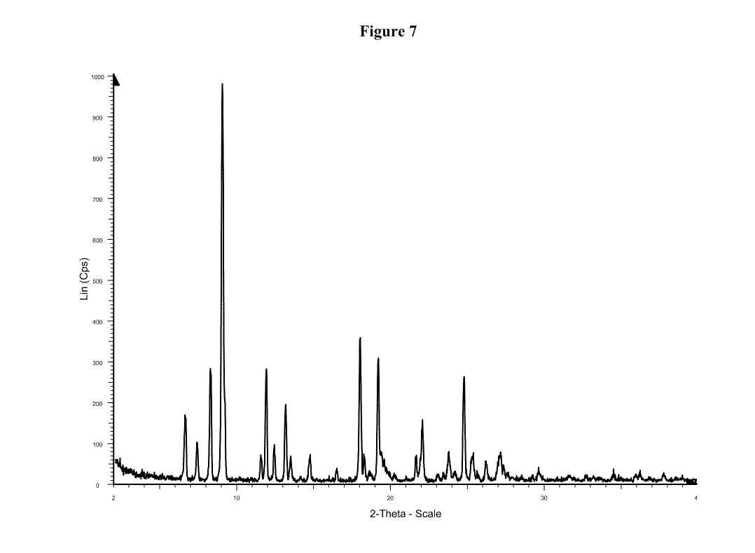

FIG. 7 is an XRPD pattern of the 2-[4-(7-ethyl-5H-pyrrolo[2,3-b]pyrazin-6-yl)-phenyl]-propan-2-ol crystalline Form C. The data show that the Form C crystal structure is unique compared to the crystal structures of the 2-[4-(7-ethyl-5H-pyrrolo[2,3-b]pyrazin-6-yl)-phenyl]-propan-2-ol Form A and Form B crystal forms.

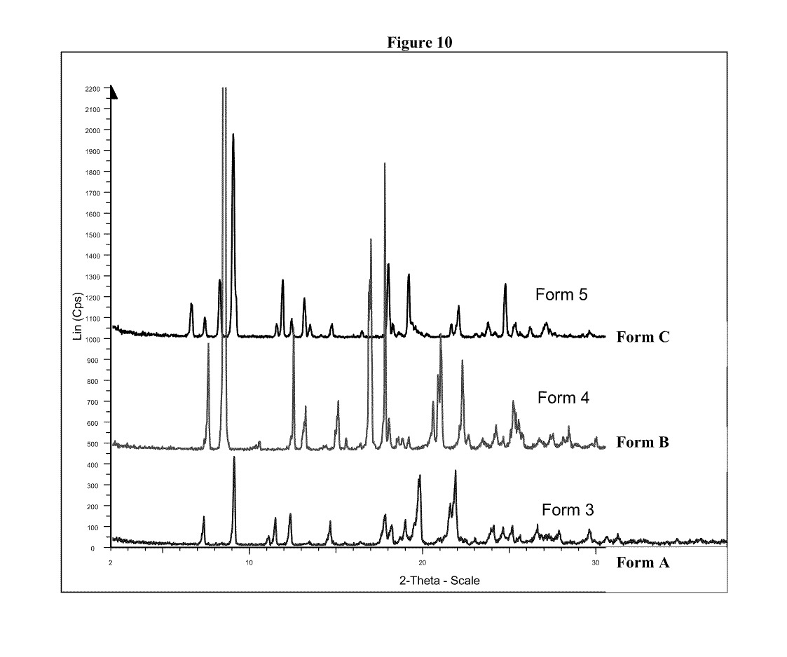

FIG. 10 is an overlay of the XRPD patterns for 2-[4-(7-ethyl-5H-pyrrolo[2,3-b]pyrazin-6-yl)-phenyl]-propan-2-ol crystalline Form A, Form B, and Form C.

A person skilled in the art will recognize that the peak locations could be slightly affected by differences in sample height. The peak locations described herein are thus subject to a variation of plus or minus (+/−) 0.15 degrees 2-theta. The relative intensities may change depending on the crystal size and morphology.

Table 1 sets forth the characteristic peak locations, d-spacings and relative intensities for the powder x-ray diffraction pattern for 2-[4-(7-ethyl-5H-pyrrolo[2,3-b]pyrazin-6-yl)-phenyl]-propan-2-ol crystalline Form A.

TABLE 1

| Measured Angle | | |
|---|---|---|
| Degrees 2θ | | |
| (+/−) 0.15 degrees | Calculated d Spacing | Relative Intensity |
| 2-theta | Angstrom | % |
| 7.30 | 12.08 | 35.0 |
| 9.09 | 9.71 | 100.0 |
| 11.08 | 7.97 | 9.1 |
| 11.47 | 7.70 | 31.9 |
| 12.34 | 7.16 | 36.4 |
| 13.22 | 6.69 | 4.1 |
| 14.67 | 6.03 | 27.8 |
| 16.50 | 5.36 | 4.3 |
| 17.83 | 4.96 | 37.9 |
| 18.20 | 4.86 | 25.5 |
| 19.01 | 4.66 | 25.5 |
| 19.56 | 4.53 | 29.8 |
| 19.82 | 4.47 | 79.4 |
| 20.99 | 4.22 | 8.8 |
| 21.60 | 4.10 | 50.0 |
| 21.91 | 4.05 | 89.9 |
| 23.06 | 3.85 | 7.4 |
| 24.04 | 3.69 | 21.0 |
| 24.67 | 3.60 | 21.8 |
| 25.21 | 3.52 | 23.3 |
| 25.64 | 3.47 | 10.5 |
| 26.66 | 3.34 | 20.0 |
| 27.95 | 3.18 | 16.7 |
| 29.72 | 3.00 | 19.5 |
| 30.74 | 2.90 | 13.0 |
| 31.35 | 2.85 | 11.7 |
| 32.88 | 2.72 | 6.0 |

In particular, the peaks at 7.30, 9.09, 11.08, 11.47, 12.34, and 14.67 (expressed in degrees 2-theta +/−0.15 degree) are characteristic of 2-[4-(7-ethyl-5H-pyrrolo[2,3-b]pyrazin-6-yl)-phenyl]-propan-2-ol crystalline Form A.

Table 2 sets forth the characteristic peak locations, d-spacings and relative intensities for the powder x-ray diffraction pattern for 2-[4-(7-ethyl-5H-pyrrolo[2,3-b]pyrazin-6-yl)-phenyl]-propan-2-ol crystalline Form B.

TABLE 2

| Measured Angle | | |
|---|---|---|
| Degrees 2θ | | |
| (+/−) 0.15 degrees | Calculated d Spacing | Relative Intensity |
| 2-theta | Angstrom | % |
| 7.50 | 11.77 | 5.7 |
| 8.45 | 10.46 | 100.0 |
| 10.47 | 8.44 | 0.5 |
| 12.46 | 7.10 | 6.5 |
| 13.11 | 6.75 | 1.5 |
| 14.29 | 6.19 | 0.2 |
| 15.03 | 5.89 | 2.6 |
| 15.50 | 5.71 | 0.7 |
| 16.33 | 5.42 | 0.4 |
| 16.90 | 5.24 | 9.1 |
| 17.78 | 4.99 | 15.2 |
| 18.01 | 4.92 | 1.7 |
| 18.50 | 4.79 | 0.6 |
| 18.78 | 4.72 | 0.6 |
| 20.54 | 4.32 | 2.6 |
| 21.02 | 4.22 | 6.2 |
| 22.27 | 3.99 | 4.8 |
| 22.59 | 3.93 | 0.8 |
| 23.42 | 3.80 | 0.7 |
| 24.20 | 3.67 | 1.4 |
| 25.23 | 3.53 | 2.5 |
| 25.73 | 3.46 | 0.9 |
| 26.74 | 3.33 | 0.7 |
| 27.45 | 3.25 | 0.7 |
| 28.12 | 3.17 | 0.7 |
| 28.43 | 3.14 | 1.3 |
| 30.01 | 2.98 | 0.7 |
| 31.33 | 2.85 | 0.8 |
| 32.39 | 2.76 | 1.0 |
| 33.20 | 2.70 | 0.4 |

In particular, the peaks at 7.50, 8.45, 12.46, 13.11, 15.03, 16.90, and 17.78 (expressed in degrees 2-theta +/−0.15 degree) are characteristic of 2-[4-(7-ethyl-5H-pyrrolo[2,3-b]pyrazin-6-yl)-phenyl]-propan-2-ol crystalline Form B.

Table 3 sets forth the characteristic peak locations, d-spacings and relative intensities for the powder x-ray diffraction pattern for 2-[4-(7-ethyl-5H-pyrrolo[2,3-b]pyrazin-6-yl)-phenyl]-propan-2-ol crystalline Form C.

TABLE 3

| Measured Angle | | |
|---|---|---|
| Degrees 2θ | Calculated | |
| (+/−) 0.15 degrees | d Spacing | Relative Intensity |
| 2-theta | Angstrom | % |
| 6.58 | 13.43 | 17.1 |
| 7.35 | 12.02 | 10.2 |
| 8.23 | 10.73 | 28.7 |
| 9.01 | 9.81 | 100.0 |
| 11.53 | 7.67 | 7.0 |
| 11.87 | 7.45 | 28.6 |
| 12.39 | 7.14 | 9.6 |
| 13.12 | 6.74 | 19.6 |
| 13.46 | 6.58 | 6.7 |
| 14.11 | 6.27 | 1.6 |
| 14.72 | 6.01 | 7.1 |
| 16.47 | 5.38 | 3.7 |
| 18.01 | 4.92 | 36.4 |
| 18.30 | 4.84 | 6.7 |
| 18.63 | 4.76 | 2.9 |
| 19.18 | 4.62 | 31.3 |
| 20.26 | 4.38 | 1.7 |
| 21.65 | 4.10 | 6.5 |
| 22.08 | 4.02 | 15.9 |
| 23.09 | 3.85 | 2.2 |
| 23.44 | 3.79 | 2.6 |
| 23.79 | 3.74 | 7.8 |
| 24.19 | 3.68 | 2.9 |
| 24.79 | 3.59 | 26.6 |
| 25.34 | 3.51 | 6.7 |
| 26.23 | 3.39 | 5.5 |
| 27.16 | 3.28 | 7.7 |
| 29.26 | 3.05 | 1.9 |
| 29.67 | 3.01 | 3.9 |
| 32.75 | 2.73 | 2.0 |
| 33.26 | 2.69 | 1.7 |
| 34.56 | 2.59 | 3.5 |
| 36.01 | 2.49 | 2.2 |
| 36.30 | 2.47 | 3.2 |
| 36.91 | 2.44 | 1.5 |
| 37.84 | 2.38 | 2.5 |

In particular, the peaks at 6.58, 7.35, 8.23, 9.01, 11.87, and 13.12 (expressed in degrees 2-theta +/−0.15 degree) are characteristic of 2-[4-(7-ethyl-5H-pyrrolo[2,3-b]pyrazin-6-yl)-phenyl]-propan-2-ol crystalline Form C.

### Raman Spectroscopy

Raman Spectrocopy is performed using a JY Horiba LabRAM Raman spectrophotmeter equipped with an Olympus BX41 Raman microscope. The sample powders are placed on a standard glass microscope slide for analysis. Raman spectra are obtained using a laser (Topica photonics Xtra wavelength stabilized laser source) at 784.86 nm and a spectrophotometer grating of 600. The material is scanned from 200 cm⁻¹ to 1800 cm⁻¹ in 400 cm⁻¹ increments. Raman spectral data was acquired for 5 seconds in each 400 cm⁻¹ increment, 15 times.

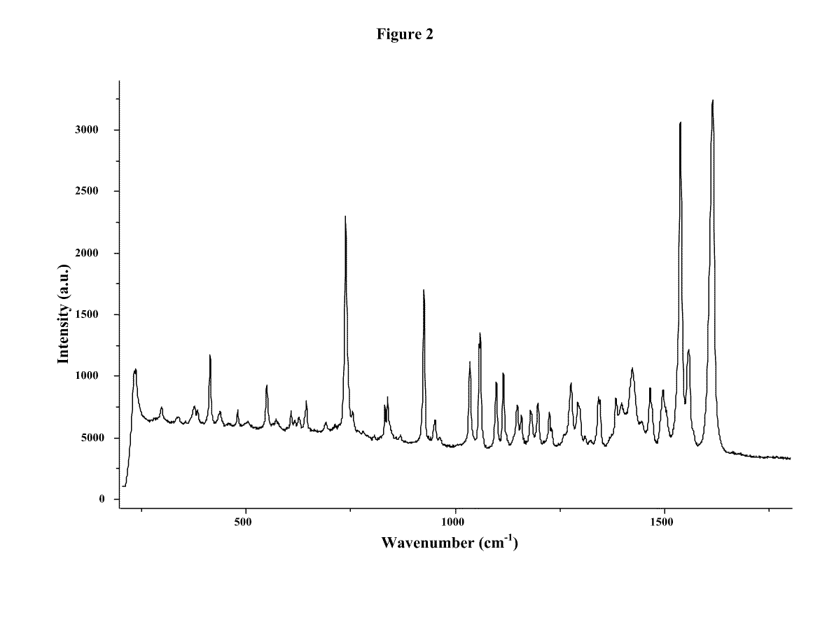

The Raman spectrum (FIG. 2) is representative of crystalline 2-[4-(7-ethyl-5H-pyrrolo[2,3-b]pyrazin-6-yl)-phenyl]-propan-2-ol Form A.

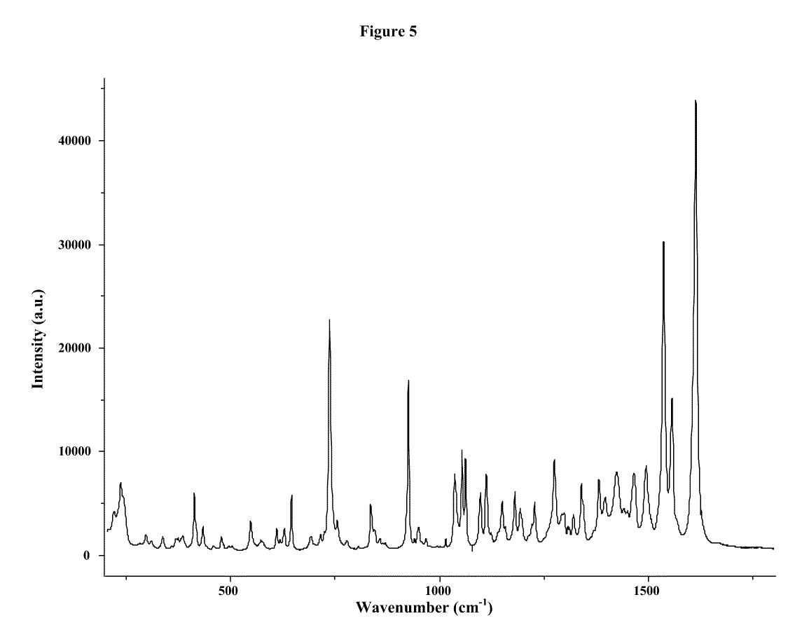

Based on the Raman spectra, the 2-[4-(7-ethyl-5H-pyrrolo[2,3-b]pyrazin-6-yl)-phenyl]-propan-2-ol crystalline Form B (FIG. 5) has a unique Raman spectrum compared to Form A.

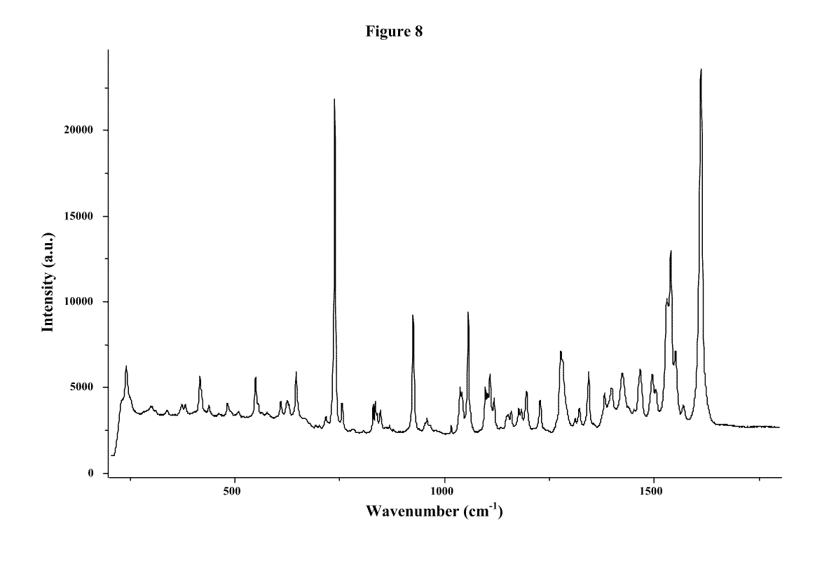

Based on the Raman spectra, the 2-[4-(7-ethyl-5H-pyrrolo[2,3-b]pyrazin-6-yl)-phenyl]-propan-2-ol crystalline Form C (FIG. 8) has a unique Raman spectrum compared to Form A and Form B.

Table 4 sets forth the characteristic peak locations for the Raman spectra of 2-[4-(7-ethyl-5H-pyrrolo[2,3-b]pyrazin-6-yl)-phenyl]-propan-2-ol crystalline Form A, Form B, and Form C.

TABLE 4

| Form A | Form B | Form C |
|---|---|---|
| Measured | Measured | Measured |
| wavenumber (cm⁻) | wavenumber (cm⁻) | wavenumber (cm⁻) |
| (±1 cm⁻¹) | (±1 cm⁻¹) | (±1 cm⁻¹) |
| 232.9 | 232.9 | 236.8 |
| 294.2 | 291.9 | 412.6 |
| 373.0 | 333.0 | 479.0 |
| 410.4 | 380.8 | 546.2 |
| 434.4 | 408.9 | 605.7 |
| 476.8 | 429.0 | 622.2 |
| 547.0 | 473.8 | 643.1 |
| 604.3 | 543.3 | 435.3 |
| 641.0 | 625.1 | 753.0 |
| 734.8 | 642.4 | 827.2 |
| 829.0 | 733.5 | 832.3 |
| 835.7 | 831.9 | 844.5 |
| 922.8 | 922.8 | 922.5 |
| 948.9 | 947.6 | 955.8 |
| 1032.3 | 1033.0 | 1035.0 |
| 1057.4 | 1050.4 | 1054.4 |
| 1095.4 | 1059.4 | 1106.2 |
| 1112.7 | 1094.7 | 1157.4 |
| 1146.2 | 1109.2 | 1175.0 |
| 1177.6 | 1147.6 | 1194.0 |
| 1195.2 | 1177.5 | 1226.3 |
| 1222.2 | 1190.4 | 1275.4 |
| 1274.4 | 1225.5 | 1320.0 |
| 1290.4 | 1273.1 | 1342.5 |
| 1340.0 | 1318.9 | 1380.6 |
| 1382.0 | 1338.0 | 1396.9 |
| 1421.6 | 1380.4 | 1423.6 |
| 1464.2 | 1395.4 | 1465.6 |
| 1494.9 | 1422.1 | 1494.5 |
| 1536.3 | 1464.2 | 1538.4 |
| 1555.2 | 1493.1 | 1610.9 |
| 1612.9 | 1535.2 | |
| | 1555.5 | |
| | 1612.8 | |

### Differential Scanning Calorimetry (DSC)

Thermal analysis of the compounds of the invention is performed using a TA Instruments Model Q-1000 Differential Scanning calorimeter under a dry nitrogen atmosphere (50 mL/min). The DSC temperature and heat flow are calibrated using an indium standard. The compound powder is transferred to an aluminum pan (TA Instruments part number 900793.901). The thermogram is acquired at a linear heating rate of 10° C. per minute.

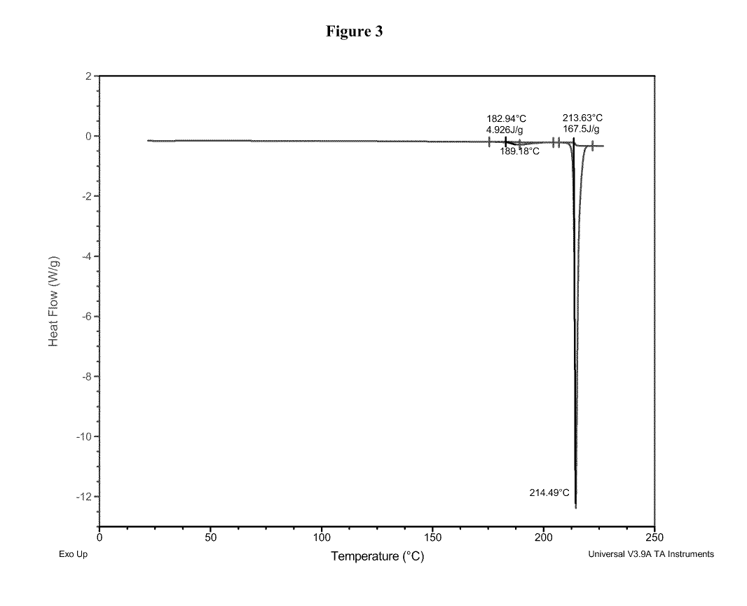

Thermal analysis for the difumarate Form A (FIG. 3) shows a broad endotherm at onset about 183° C. which is the sublimation of Form A to Form B. The in-situ prepared Form B phase, created by sublimation of Form A, melts at about 214° C. under these conditions.

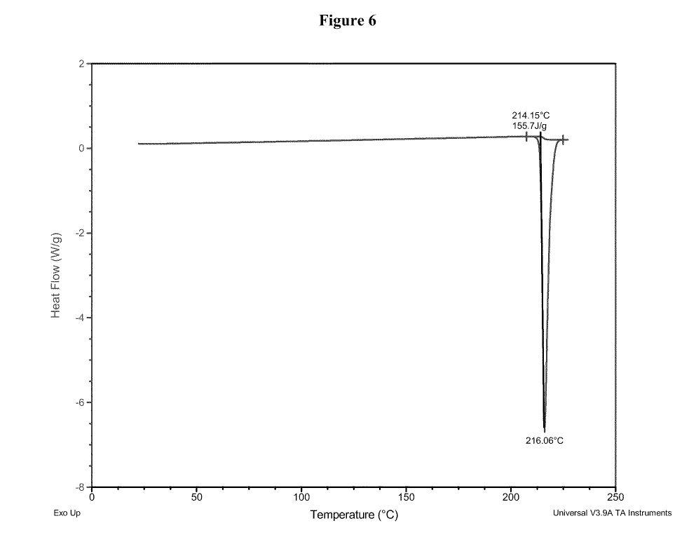

FIG. 6 is an overlay of the thermal profile of the Form B. The DSC of the Form B shows the melt of the crystalline phase at an onset of about 214° C.

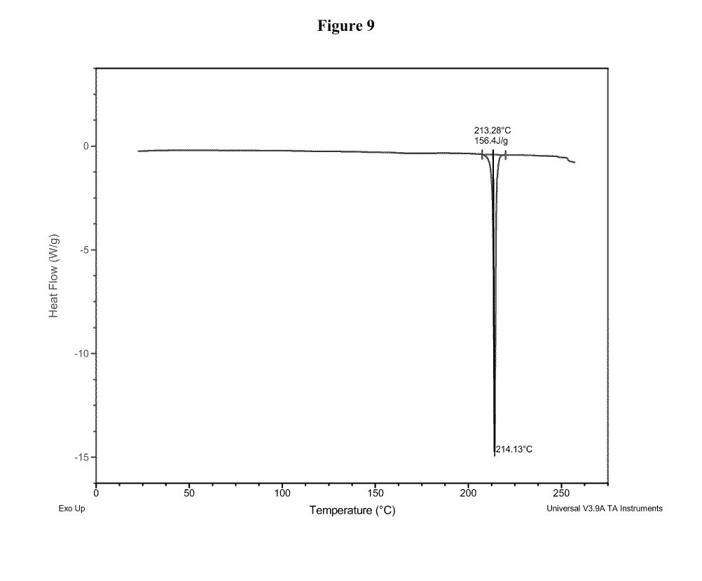

FIG. 9 is an overlay of the thermal profile of the Form C. The DSC of the Form C shows the melt of the crystalline phase at an onset of about 213° C.

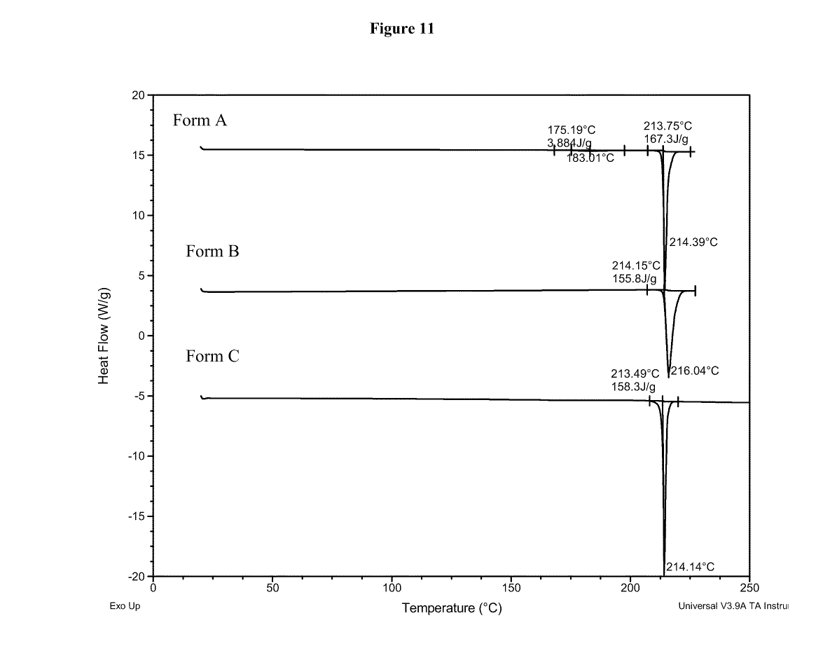

FIG. 11 is an overlay of the Form A, Form B and Form C, DSC thermograms.

What is claimed is:

1. A crystalline form of Compound I: 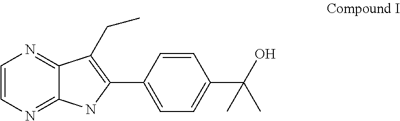
which is crystalline Form A.

2. The compound according to claim 1 exhibiting at least two significant peaks in the X-ray diffraction pattern chosen from the following list of at about 7.30, 9.09, 11.08, 11.47, 12.34, and 14.67 degrees 2-theta.

3. The compound according to claim 1 exhibiting at least three significant peaks in the X-ray diffraction pattern chosen from the following list of at about 7.30, 9.09, 11.08, 11.47, 12.34, and 14.67 degrees 2-theta.

4. The compound according to claim 1 exhibiting significant peaks in the X-ray diffraction at about 7.30, 9.09, 11.08, 11.47, 12.34, and 14.67 degrees 2-theta.

5. A crystalline form of Compound I 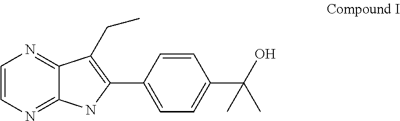
which is crystalline Form B.

6. The compound according to claim 5 exhibiting at least two significant peaks in the X-ray diffraction pattern chosen from the following list of at about 7.50, 8.45, 12.46, 13.11, 15.03, 16.90, and 17.78 degrees 2-theta.

7. The compound according to claim 5 exhibiting at least three significant peaks in the X-ray diffraction pattern chosen from the following list of at about 7.50, 8.45, 12.46, 13.11, 15.03, 16.90, and 17.78 degrees 2-theta.

8. The compound according to claim 5 exhibiting significant peaks in the X-ray diffraction at about 7.50, 8.45, 12.46, 13.11, 15.03, 16.90, and 17.78 degrees 2-theta.

9. A crystalline form of Compound I 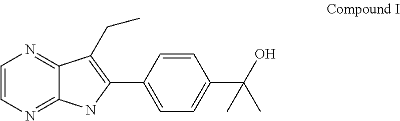
which is Form C.

10. The compound according to claim 9 exhibiting at least two significant peaks in the X-ray diffraction pattern chosen from the following list of at about 6.58, 7.35, 8.23, 9.01, 11.87, and 13.12 degrees 2-theta.

11. The compound according to claim 9 exhibiting at least three significant peaks in the X-ray diffraction pattern chosen from the following list of at about 6.58, 7.35, 8.23, 9.01, 11.87, and 13.12 degrees 2-theta.

12. The compound according to claim 9 exhibiting significant peaks in the X-ray diffraction at about 6.58, 7.35, 8.23, 9.01, 11.87, and 13.12 degrees 2-theta.

13. A pharmaceutical composition comprising a compound selected from the group consisting of crystalline Form A, Form B and Form C of Compound I: 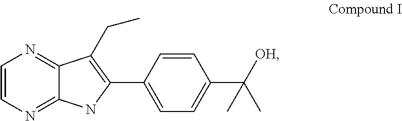
together with at least one pharmaceutically acceptable carrier or excipient.

14. A method of treating a condition in a patient selected from joint inflammation, rheumatoid arthritis, a tumor, and mantle cell lymphoma, comprising administering to a patient in need thereof an effective amount of a a compound selected from the group consisting of crystalline Form A, Form B and Form C of Compound I:

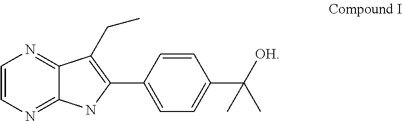

15. The method according to claim 14, wherein the condition is joint inflammation.

16. The method according to claim 15, wherein the compound is administered in combination with methotrexate.

17. The method according to claim 14 wherein the condition is rheumatoid arthritis.

18. The method according to claim 17, wherein the compound is administered in combination with methotrexate.

19. The method according to claim 14 wherein the condition is a tumor.

20. The method according to claim 14 wherein the condition is a mantle cell lymphoma.

21. A method of inhibiting angiogenesis in a patient, comprising administering to a patient in need thereof an effective amount of a compound selected from the group consisting of crystalline Form A, Form B and Form C of Compound I:

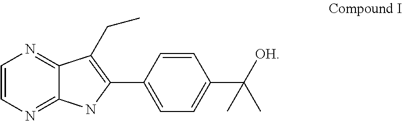

22. A method of making Form A according to claim 1, comprising crystallizing Compound I from n-propanol 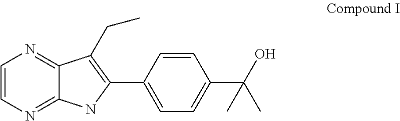
to give a form which has substantially the XRPD pattern of FIG. 1.

23. A method of making Form B according to claim 5, comprising crystallizing Compound I from n-propanol to give a form which has substantially the XRPD pattern of Form B of FIG. 4.

24. A method of making Form C according to claim 9, comprising crystallizing Compound I from methyltetrahydrofuran to give a form which has substantially the XRPD pattern of FIG. 7.
